(19) United States
(12) Patent Application Publication
(10) Pub. No.: US 2013/0027662 A1
(43) Pub. Date: Jan. 31, 2013

(54) OPHTHALMOLOGIC APPARATUS

(71) Applicant: Ryutaro Matsuoka, Tokyo (JP)

(72) Inventor: Ryutaro Matsuoka, Tokyo (JP)

(73) Assignee: CANON KABUSHIKI KAISHA, Tokyo (JP)

(21) Appl. No.: 13/558,103

(22) Filed: Jul. 25, 2012

(30) Foreign Application Priority Data
Jul. 29, 2011 (JP) 2011-167050

(51) Int. Cl. A61B 3/14

(52) U.S. Cl. 351/206

(57) ABSTRACT

An ophthalmologic apparatus includes a first casing which includes a light source for emitting illumination light including at least one of ultraviolet light and far-infrared light so as to illuminate a fundus of an eye to be examined, and an attenuation unit attenuating at least one of the ultraviolet light and the far-infrared light included in the illumination light, and a second casing which includes at least a part of an illumination optical system for guiding the illumination light attenuated by the attenuation unit to the fundus or a part of an imaging optical system for guiding the light reflected from the fundus to an imaging unit for imaging the fundus, and which is formed of a material lighter than the material forming the first casing.

### BACKGROUND OF THE INVENTION

1. Field of the Invention

The present invention relates to an ophthalmologic apparatus.

2. Description of the Related Art

An existing ophthalmologic apparatus includes a head unit with an optical system for observing and imaging a fundus of an eye to be examined and a base unit which is provided in the lower portion of the head unit to support the head unit. The head unit includes a light source and a plurality of optical members in a casing inside an external cover, and these are appropriately arranged so that the illumination light emitted from the light source is incident to the eye to be examined at the time of observing and imaging the fundus of the eye to be examined.

In the ophthalmologic apparatus with the above-described configuration, the temperature inside the casing increases with the emission of light from the light source due to a heat ray included in the illumination light emitted from the light source. In order to suppress an increase in temperature inside the casing, there is a known ophthalmologic apparatus which includes a mechanism for highly efficiently radiating the accumulated heat as discussed in Japanese Patent Application Laid-Open No. 04-364826. Further, the illumination light also includes an ultraviolet ray.

With the above-described reasons, the casing of the head unit is generally formed of, for example, a metal material which has a melting point higher than that of a plastic material and is hardly degraded due to an ultraviolet ray.

However, an issue arises in that the ophthalmologic apparatus becomes heavier due to the use of the metal material for the casing of the head unit.

### SUMMARY OF THE INVENTION

Aspects of the present invention are directed to an ophthalmologic apparatus capable of decreasing the weight of an ophthalmologic apparatus. Furthermore, the invention is not limited to the above-described object, and aspects of the present invention are also directed to an ophthalmologic apparatus capable of achieving the operation and the effect, which are obtained by the respective configurations illustrated in the exemplary embodiments to be described below and are not obtained by the related art.

According to an aspect of the present invention, an ophthalmologic apparatus includes a light source which emits illumination light including at least one of ultraviolet light and far-infrared light so as to illuminate a fundus of an eye to be examined, an attenuation unit which attenuates at least one of the ultraviolet light and the far-infrared light included in the illumination light, an illumination optical system which guides the illumination light attenuated by the attenuation unit to the fundus, an imaging optical system which guides the light reflected from the fundus to an imaging unit imaging the fundus, a first casing which includes at least the light source and the attenuation unit, and a second casing which includes at least a part of the illumination optical system or a part of the imaging optical system, and which is formed of a material lighter than the material forming the first casing.

Further features and aspects of the present invention will become apparent from the following detailed description of exemplary embodiments with reference to the attached drawings.

### BRIEF DESCRIPTION OF THE DRAWINGS

The accompanying drawings, which are incorporated in and constitute a part of the specification, illustrate exemplary embodiments, features, and aspects of the invention and, together with the description, serve to explain the principles of the invention.

FIG. 1 is a diagram illustrating an example of an entire ophthalmologic apparatus.

FIG. 2 is a diagram illustrating a configuration example of a head unit of the ophthalmologic apparatus.

FIGS. 3A and 3B are diagrams respectively illustrating light emission spectrum distribution examples of a xenon tube and an infrared light emitting diode.

FIGS. 4A and 4B are diagrams respectively illustrating optical characteristic examples of an ultraviolet-infrared (UV-IR) cutoff filter and a dichroic mirror.

FIG. 5 is a diagram illustrating an example of a casing of the ophthalmologic apparatus.

### DESCRIPTION OF THE EMBODIMENTS

Various exemplary embodiments, features, and aspects of the invention will be described in detail below with reference to the drawings.

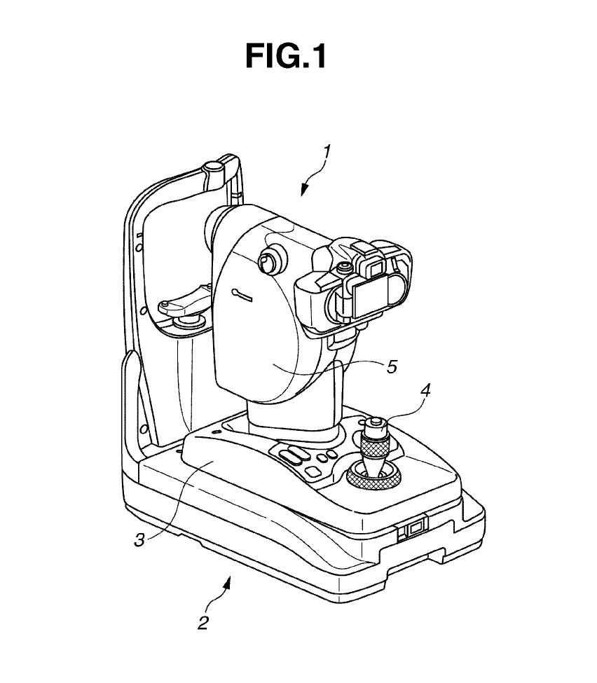

Hereinbelow, referring to FIGS. 1 to 5, an exemplary embodiment will be described. However, the discussed technique is not limited to the following exemplary embodiment, and may be implemented in various modified forms without departing from the spirit of the exemplary embodiment.

FIG. 1 is a diagram illustrating an example of an entire ophthalmologic apparatus. A head unit 1 equipped with an optical system for observing and imaging a fundus of an eye to be examined is covered by an external cover 5, and is fixed to a movable stage 3 which is movable on a base unit 2 forward, backward, leftward, and rightward. At the time of observing and imaging the fundus, an examiner can adjust the position of the head unit 1 relative to the eye to be examined by operating an operation stick 4 provided in the movable stage 3.

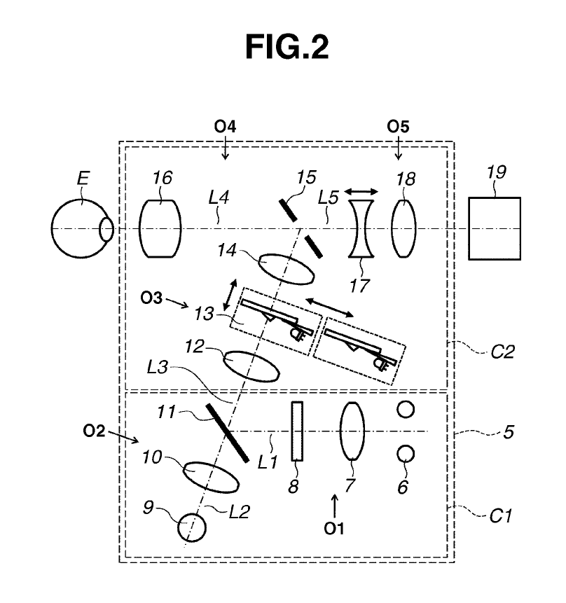

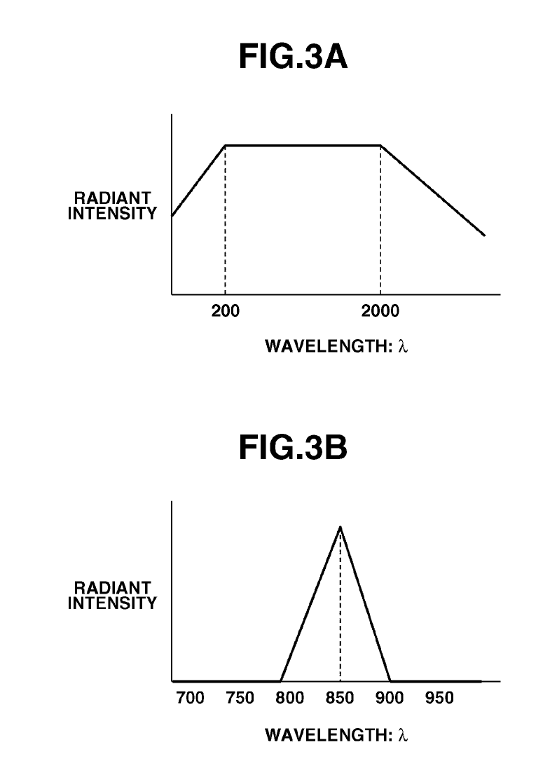

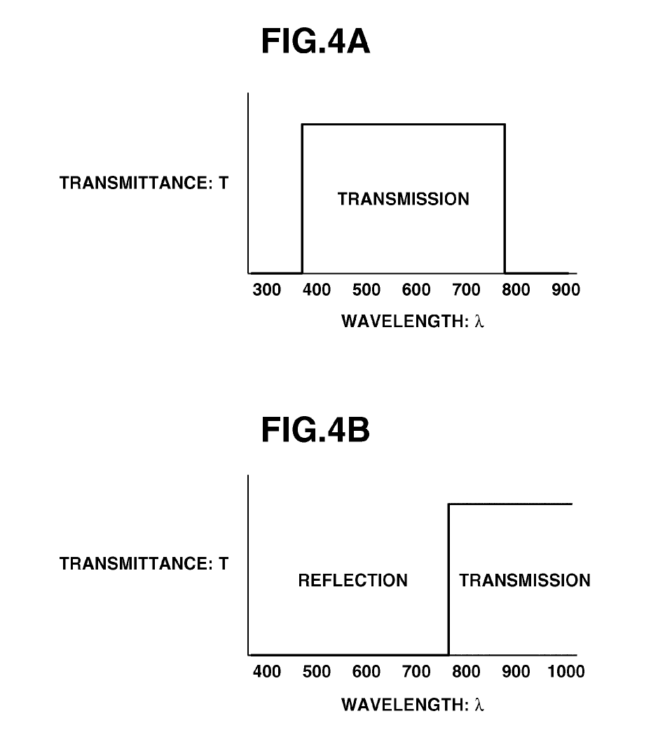

FIG. 2 is a diagram illustrating a configuration example of the head unit 1 of the ophthalmologic apparatus. Specifically, FIG. 2 is a diagram illustrating a configuration example of the inside of the external cover 5. Further, FIGS. 3A and 3B are diagrams respectively illustrating light emission spectrum distribution examples of a xenon tube and an infrared light emitting diode. Furthermore, FIGS. 4A and 4B are diagrams respectively illustrating optical characteristic examples of a UV-IR cutoff filter 8 and a dichroic mirror 11.

As illustrated in FIG. 2, the head unit of the ophthalmologic apparatus includes an imaging light source unit O1, an observation light source unit O2, an illumination optical system O3, an illuminating and imaging optical system O4, and an imaging optical system O5.

The imaging light source unit O1 includes a xenon tube 6, a condenser lens 7, and a UV-IR cutoff filter 8. The xenon tube 6, which is an imaging light source, and has a light emission spectrum distribution illustrated in FIG. 3A, the condenser lens 7 which focuses the light emitted from the xenon tube 6, and the UV-IR cutoff filter 8 which has optical characteristics illustrated in FIG. 4A are sequentially arranged on the optical axis L1 in the light traveling direction.

Furthermore, as the imaging light source, for example, a halogen lamp, an incandescent lamp, or the like may be used instead of the xenon tube 6. Further, as illustrated in FIG. 3A, the illumination light emitted from the xenon tube 6 includes ultraviolet light and far-infrared light.

Herein, a heat ray (far-infrared light) is, for example, an electromagnetic wave having a wavelength band of about 3000 nm to 1 mm, and becomes a factor causing thermal deformation due to an increase in temperature when the heat ray is irradiated to plastic. Further, an ultraviolet ray (ultraviolet light) is, for example, an electromagnetic wave having a wavelength band of about 1 nm to 380 nm, and becomes a factor causing ultraviolet ray degradation when the ultraviolet ray is irradiated to plastic.

Further, the illumination light emitted from the xenon tube 6 includes ultraviolet light and far-infrared light, but the invention is not limited thereto. For example, the illumination light may include only any one of ultraviolet light and far-infrared light. Accordingly, the imaging light source is not limited to the xenon tube 6.

Herein, the xenon tube 6 corresponds to an example of a light source which emits illumination light including at least one of ultraviolet light and far-infrared light so as to illuminate the fundus of the eye to be examined. Further, the condenser lens 7 corresponds to an example of a lens which condenses the illumination light.

As illustrated in FIG. 4A, the UV-IR cutoff filter 8 has a characteristic in which ultraviolet light and infrared light are attenuated by the UV-IR cutoff filter. Furthermore, since far-infrared light and ultraviolet light do not contribute to the imaging and illuminating of the fundus, any issue does not occur at the time of imaging the fundus even when these are attenuated.

Further, when the xenon tube 6 includes only any one of ultraviolet light and far-infrared light, a UV cutoff filter or an IR cutoff filter may be used instead of the UV-IR cutoff filter 8.

That is, the UV-IR cutoff filter 8 or the like corresponds to an example of an attenuation unit which attenuates at least one of ultraviolet light and far-infrared light included in the illumination light and a bandwidth filter which attenuates at least one of ultraviolet light and far-infrared light included in the illumination light. Further, the characteristics of the UV-IR cutoff filter 8 are not limited to those illustrated in FIG. 4A.

The observation light source unit O2 includes an infrared light emitting diode 9 and a condenser lens 10. The infrared light emitting diode 9 which is an observation light source and has a light emission spectrum distribution illustrated in FIG. 3B, and a condenser lens 10 which condenses the light emitted from the infrared light emitting diode 9 are sequentially arranged on the optical axis L2 in the light traveling direction.

Herein, it is described that the infrared light emitting diode 9 emitting near-infrared light has a light emission spectrum distribution illustrated in FIG. 3B, but the invention is not limited thereto. As long as the infrared light emitting diode 9 emits near-infrared light, the infrared light emitting diode 9 does not have the light emission spectrum distribution illustrated in FIG. 3B.

Furthermore, as an observation light source, a halogen lamp may be used instead of the infrared light emitting diode 9, by removing visible light and infrared light (mid-infrared light and far-infrared light) from the light emitting flux of the halogen lamp using an optical filter, and extracting near-infrared light. That is, the observation light source is not limited to the infrared light emitting diode 9.

The illumination optical system O3 includes the dichroic mirror 11, a first relay lens 12, and a second relay lens 14. The dichroic mirror 11 having the optical characteristics illustrated in FIG. 4B, the first relay lens 12, a split index unit 13 for a focus adjustment, and the second relay lens 14 are sequentially arranged on the optical axis L3 in the light traveling direction.

The split index unit 13 includes a movement mechanism which can move a split index along the optical axis L3 and an insertion and separation mechanism which can insert the split index into the optical axis L3 or can separate the split index from the optical axis L3, and is controlled by a control unit (not illustrated).

Further, as illustrated in FIG. 4B, the dichroic mirror 11 has a characteristic in which visible light is reflected therefrom and infrared light is transmitted therethrough. Furthermore, the characteristics of the dichroic mirror 11 are not limited to those illustrated in FIG. 4B.

The illuminating and imaging optical system O4 includes a perforated mirror 15 and an objective lens 16. The perforated mirror 15 with a hole formed at the center of a mirror and the objective lens 16, are sequentially arranged on the optical axis L4 in the traveling direction of the light incident to the eye to be examined.

The imaging optical system O5 includes a focus lens 17, an imaging lens 18, and an imaging unit 19. The focus lens 17, the imaging lens 18, and the imaging unit 19 are sequentially arranged on the optical axis L5 in the light traveling direction. The focus lens 17 includes a movement mechanism which moves the focus lens along the optical axis L5, and may adjust the focus of the fundus image which is formed on the imaging unit 19.

Furthermore, as illustrated in FIG. 2, the dichroic mirror 11 which is included in the imaging light source unit O1, the observation light source unit O2, and the illumination optical system O3 is disposed as described above inside a first casing C1 which is formed of a metal material.

Further, as illustrated in FIG. 2, the member of the illumination optical system O3 excluding the dichroic mirror 11, the illuminating and imaging optical system O4, and the imaging optical system O5 excluding the imaging unit 19 are arranged as described above inside, for example, a second casing C2, which is formed of a plastic material.

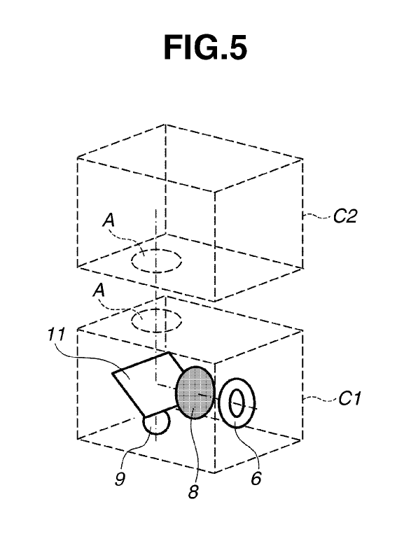

FIG. 5 is a diagram schematically illustrating configurations of the first casing C1 and the second casing C2 to be described below. As illustrated in FIG. 5, the first casing C1 and the second casing C2 are respectively equipped with an aperture portion A. Through the aperture portion A, visible light which is reflected by the dichroic mirror 11 or near-infrared light which is transmitted through the dichroic mirror 11 can be incident to the second casing C2.

Furthermore, in FIG. 5, the condenser lenses 7 and 10 inside the first casing C1 and the respective members inside the second casing C2 are omitted for the simplicity of description. Furthermore, in order to clarify the aperture portion A, the first casing C1 and the second casing C2 are separated from each other.

Further, since the light emitted from the xenon tube 6 is condensed by the condenser lens 7 and is incident to the UV-IR cutoff filter 8, ultraviolet light and far-infrared light are incident to the second casing C2 through the aperture portion A, thereby suppressing ultraviolet ray degradation and thermal deformation.

That is, since the light emitted from the xenon tube 6 is condensed by the condenser lens 7 and is incident to the UV-IR cutoff filter 8, ultraviolet light or far-infrared light is efficiently attenuated, and the diffusion of ultraviolet light or far-infrared light is prevented.

Herein, the material forming the second casing is not limited to the plastic, and a material lighter than the metal forming the first casing can be used. Furthermore, in one case the material forming the second casing can be easily molded compared to the material forming the first casing. That is, it may be the case that the second casing is formed of a material which is lighter than the metal material and is more easily molded.

Furthermore, although it is described that the dichroic mirror 11 included in the imaging light source unit O1, the observation light source unit O2, and the illumination optical system O3 is provided inside the first casing C1, the invention is not limited thereto. The first casing C1 formed of the metal material may include therein at least a source for generating the ultraviolet ray and the heat ray and an attenuation unit for attenuating the ultraviolet ray and the heat ray so that the ultraviolet ray and the heat ray are not incident to the second casing C2.

For example, at least the xenon tube 6 emitting the ultraviolet ray and the heat ray, and the UV-IR cutoff filter 8 may be provided inside the first casing C1, and other members may be appropriately provided inside the first casing C1 or the second casing C2. That is, the first casing C1 corresponds to an example of the first casing which includes at least the light source and the attenuation unit and is formed of a metal material.

Further, it is described that the members of the illumination optical system O3 excluding the dichroic mirror 11, the illuminating and imaging optical system O4, and the imaging optical system O5 excluding the imaging unit 19 are provided inside the second casing C2, but the invention is not limited thereto.

For example, at least a part of the observation light source unit O2, the illumination optical system O3, the members, the illuminating and imaging optical system O4, and the imaging optical system O5 excluding the imaging unit 19 may be provided inside the second casing C2. That is, the second casing C2 corresponds to the second casing which includes at least a part of the illumination optical system or a part of the imaging optical system and is formed of a material lighter than the metal material.

Furthermore, for example, when the xenon tube 6 emitting the ultraviolet ray and the heat ray, and the UV-IR cutoff filter 8 are provided inside the first casing C1, and other members are provided inside the second casing C2, the plastic material may be used more, thereby further decreasing the weight of the ophthalmologic apparatus.

Further, the second casing C2 is provided in the upper portion of the first casing C1, and the first and second casings are both covered by the external cover 5 of the head unit 1 illustrated in FIG. 1.

Furthermore, the “casing” used in the present exemplary embodiment indicates a single component accommodating one or more constituting components or a plurality of components combined with each other to form the single component, and does not include a component, for example, a cover covering the opening of the casing or a screw fixing the casing.

Further, the “metal material” used in the present exemplary embodiment indicates a material in which a material other than metal is included in metal and which has a melting point at which the degradation caused by the ultraviolet ray hardly occurs (and may not even occur at all) and the shape hardly changes (and may not even change at all) due to an increase in temperature caused by the heat ray.

Furthermore, the “plastic material” used in the present exemplary embodiment indicates a material in which a material other than plastic is included in plastic and which is lighter than the metal material. Further, the “plastic material” indicates a material in which a material other than plastic is included in plastic and which is more easily molded compared to the metal material.

Next, the operation of the ophthalmologic apparatus according to aspects of the invention will be described. At the time of observing the fundus, near-infrared light emitted from the infrared light emitting diode 9 is first condensed by the condenser lens 10. The condensed near-infrared light is transmitted through the dichroic mirror 11, and is reflected by the perforated mirror 15 toward the optical axis L4 after passing through the relay lenses 12 and 14.

The reflected illumination light is irradiated to the fundus of the eye E to be examined facing the objective lens 16. The light reflected from the fundus passes through the objective lens 16, the hole of the center of the perforated mirror 15, and the focus lens 17 and the imaging lens 18, and forms an image in the imaging unit 19. The formed fundus image is photoelectrically converted by an imaging device, is A/D converted by a calculation process circuit (not illustrated), and is displayed on a display unit (not illustrated).

On the other hand, at the time of imaging the fundus, flash light emitted from the xenon tube 6 is first condensed by the condenser lens 7, and then only the light of a wavelength band, for example, from 380 nm to 780 nm passes through the UV-IR cutoff filter 8.

In other words, visible light is selected by the UV-IR cutoff filter 8. The light transmitted through the UV-IR cutoff filter 8 is reflected along the optical axis L3 by the dichroic mirror 11, and is reflected toward the optical axis L4 again by the perforated mirror 15 after passing through the relay lenses 12 and 14.

The reflected illumination light is irradiated to the fundus of the eye E facing the objective lens 16. That is, the dichroic mirror 11, the relay lenses 12 and 14, the perforated mirror 15, and the objective lens 16 correspond to an example of an illumination optical system which guides the illumination light attenuated by the attenuation unit to the fundus.

Then, the light reflected from the fundus passes through the objective lens 16 and the hole of the center of the perforated mirror 15, and forms an image in the imaging unit 19 after passing through the focus lens 17 and the imaging lens 18. That is, the objective lens 16, the perforated mirror 15, the focus lens 17, and the imaging lens 18 correspond to an example of an imaging optical system which guides the light reflected from the fundus to the imaging unit imaging the fundus.

The formed fundus image is photoelectrically converted by the imaging device, is A/D converted by the calculation process circuit, and is stored in a storage medium (not illustrated). That is, the imaging unit 19 corresponds to an example of an imaging unit imaging the fundus.

Furthermore, since the miosis of the subject is suppressed by using near-infrared light as the fundus observation illumination light, the fundus may be imaged without applying a mydriatic agent.

As described above, according to the present exemplary embodiment, the light (for example, ultraviolet light or far-infrared light) of a wavelength band unnecessary for the imaging of the fundus in the illumination light flux is selectively attenuated inside the first casing C1 formed of the metal material, and the light necessary for the imaging of the fundus is incident to the second casing C2.

That is, when the illumination light flux emitted from the xenon tube 6 as the imaging light source passes through the UV-IR cutoff filter 8 having optical characteristics illustrated in FIG. 4A and disposed inside the first casing C1, the heat ray (far-infrared ray) and the ultraviolet ray are attenuated, and are guided into the second casing C2.

Thus, since the ultraviolet ray degradation or the thermal deformation of the plastic caused by the ultraviolet light and the far-infrared light can be prevented, the casing which is formed of the plastic material lighter than the metal material can be used. Then, since the casing C2 formed of the plastic material can be used, it is possible to decrease the weight of the head unit 1, that is, the weight of the ophthalmologic apparatus.

Further, since the second casing C2 formed of the plastic material is provided in the upper portion of the first casing C1 formed of the metal material, the center of gravity of the head unit 1 is lowered, which improves the stability when the examiner moves the head unit 1.

Furthermore, since the strength of the base unit 2 may be decreased with a decrease in weight of the head unit 1, the entire ophthalmologic apparatus may be decreased in size and weight due to a decrease in size of the base unit 2.

Further, since the plastic material is a material which may be easily molded compared to the metal material, a freedom degree in design of the shape of the casing is improved. Furthermore, since the plastic material is lower in cost than the metal material, the lower cost apparatus may be provided.

Furthermore, in the present exemplary embodiment, the UV-IR cutoff filter 8 is used as a unit for removing the heat ray and the ultraviolet ray from the illumination light flux, but the unit for separating the wavelength of the illumination light flux is not limited thereto. In addition, one or a combination of optical members such as a UV cutoff filter, an IR cutoff filter, a cold mirror (cold filter), a hot mirror (filter), and a dichroic mirror (filter) can be used to separate the same wavelength.

For example, the UV cutoff filter and the IR cutoff filter may be used while being arranged on the optical axis instead of the UV-IR cutoff filter 8. Further, the UV cutoff filter may be used instead of the UV-IR cutoff filter 8, and the cold mirror may be used instead of the dichroic mirror 11. Furthermore, the light which is transmitted through the hot mirror among the light from the xenon tube 6 may be guided to the second casing without using the UV-IR cutoff filter 8.

The light emission of the xenon tube 6 may be controlled in consideration of the temperature inside the first casing C1 formed of the metal material. For example, the amount of the light emitted from the xenon tube 6 is obtained by the accumulation at each predetermined time using an integrating circuit or the like. That is, the integrating circuit integrates the illumination light emitted from the light source.

Then, a processing unit (not illustrated) such as a central processing unit (CPU) obtains the amount of heat of the xenon tube 6 from the light amount accumulated for a predetermined time. That is, the processing unit serves as an example of a heat amount determination unit which determines the amount of heat of the light source based on the integral value for a predetermined time.

Furthermore, as an example of a method of obtaining the heat amount, the processing unit obtains the heat amount by using a table in which the accumulated light amount is related to the heat amount. The processing circuit controls the light amount of the xenon tube 6 according to the heat amount. For example, when it is determined that the heat amount exceeds a predetermined threshold value, the processing unit reduces the light emission amount of the xenon tube 6.

Further, the processing unit may stop the emission of light of the xenon tube 6 instead of reducing the light emission amount of the xenon tube 6. That is, the processing unit serves as an example of a determination unit which determines whether the heat amount determined in the heat amount determination unit is larger than the predetermined threshold value. Furthermore, the processing unit serves as an example of the control unit which decreases the light amount of the illumination light emitted from the light source when the determination unit determines that the heat amount is larger than the threshold value.

Furthermore, the integrating circuit may integrate the amount of light emitted from the xenon tube 6 by accumulating a current from a photo diode, which is arranged on the optical axis of the xenon tube 6 on the opposite side of the eye to be examined, in a capacitor.

In this way, the temperature inside the first casing C1 formed of the metal material does not increase too much, and the influence on the plastic material having a melting point lower than the metal material and existing adjacent to the metal material may be reduced.

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures, and functions.

This application claims priority from Japanese Patent Application No. 2011-167050 filed Jul. 29, 2011, which is hereby incorporated by reference herein in its entirety.

What is claimed is:

1. An ophthalmologic apparatus comprising:
a light source configured to emit illumination light including at least one of ultraviolet light and far-infrared light so as to illuminate a fundus of an eye to be examined;
an attenuation unit configured to attenuate at least one of the ultraviolet light and the far-infrared light included in the illumination light;
an illumination optical system configured to guide the illumination light attenuated by the attenuation unit to the fundus;
an imaging optical system configured to guide the light reflected from the fundus to an imaging unit configured to image the fundus;
a first casing including at least the light source and the attenuation unit; and
a second casing including at least a part of the illumination optical system or a part of the imaging optical system and which is formed of a material lighter than the material forming the first casing.

2. The ophthalmologic apparatus according to claim 1, wherein the second casing is provided in the upper portion of the first casing.

3. The ophthalmologic apparatus according to claim 1, wherein the second casing is formed of a material lighter than the material forming the first casing and is more easily molded than the material forming the first casing.

4. The ophthalmologic apparatus according to claim 1, wherein the attenuation unit is a bandpass filter which attenuates at least one of the ultraviolet light and the far-infrared light included in the illumination light.

5. The ophthalmologic apparatus according to claim 4, further comprising:
a lens configured to condense the illumination light,
wherein the illumination light condensed by the lens is incident to the bandpass filter.

6. The ophthalmologic apparatus according to claim 1, wherein the second casing is formed of a plastic material.

7. The ophthalmologic apparatus according to claim 1, further comprising:
an integrating circuit configured to integrate the illumination light emitted from the light source;
a heat amount determination unit configured to determine a heat amount of the light source based on an integral value during a predetermined time;
a determination unit configured to determine whether the heat amount determined by the heat amount determination unit is larger than a predetermined threshold value; and
a control unit configured to decrease the light amount of the illumination light emitted from the light source when the determination unit determines that the heat amount is larger than the threshold value.

8. The ophthalmologic apparatus according to claim 1, wherein the first casing is formed of a metal material.
